(19) United States
(12) Patent Application Publication
(10) Pub. No.: US 2025/0108217 A1
(43) Pub. Date: Apr. 3, 2025

(54) Applying an Electrical Signal Using a Multi-Part Electrode Assembly, and Interrupting the Electrical Signal if the Parts Begin to Separate

(71) Applicant: Novocure GmbH, Root D4 (CH)

(72) Inventors: Yoram WASSERMAN, Haifa (IL); Stas OBUCHOVSKY, Haifa (IL); David SHAPIRO, Haifa (IL); Inbar SCHLACHET DRUKERMAN, Haifa (IL)

(73) Assignee: Novocure GmbH, Root D4 (CH)

(21) Appl. No.: 18/898,993

(22) Filed: Sep. 27, 2024

Related U.S. Application Data
(60) Provisional application No. 63/615,903, filed on Dec. 29, 2023.
(60) Provisional application No. 63/541,361, filed on Sep. 29, 2023.

(51) Int. Cl. A61N 1/36; A61N 1/04

(52) U.S. Cl. CPC A61N 1/36034; A61N 1/0476; A61N 1/0496; A61N 1/36031

(57) ABSTRACT

Alternating electric fields (e.g., tumor treating fields or TTFields) can be applied to a subject's body using electrode assemblies that are made from two discrete subassemblies, which are removably and adhesively connectable to each other. As long as the two subassemblies remain in intimate contact (which can be determined, e.g., by measuring an electrical resistance or an optical reflectance), the system applies an AC voltage to the electrode assemblies. But if (a) the subassemblies within any given electrode assembly become separated (or even begin to separate), or (b) the subassemblies within any given electrode assembly are not aligned correctly, then the system will turn off the AC voltage to that electrode assembly.

### CROSS REFERENCE TO RELATED APPLICATIONS

This application claims the benefit of U.S. Provisional Applications 63/541,361 (filed Sep. 29, 2023) and 63/615,903 (filed Dec. 29, 2023), each of which is incorporated herein by reference in its entirety.

### BACKGROUND

Tumor Treating Fields (TTFields) therapy is a proven approach for treating tumors using alternating electric fields at frequencies e.g., between 50 KHz-5 MHz, more commonly 100-500 KHz. Conventionally, the alternating electric fields are induced by electrode assemblies (e.g., arrays of capacitively coupled electrodes, also called transducer arrays) placed on the subject's skin on opposite sides of the subject's body. When an AC voltage is applied between opposing electrode assemblies, an AC current is coupled through the electrode assemblies and into the subject's body. And higher currents are strongly correlated with higher efficacy of treatment.

Alternating electric fields can also be used to treat medical conditions other than tumors. For example, as described in U.S. Pat. No. 10,967,167, alternating electric fields e.g., at 75-150 kHz can be used to increase the permeability of the blood brain barrier so that, e.g., chemotherapy drugs can reach the brain.

Conventional electrode assemblies for applying TTFields to a subject's body are built from a plurality of parts that are all incorporated into a single integrated unit, and the entire integrated unit is either applied to the subject's body as a whole or removed from the subject's body as a whole. Examples of such single-integrated-unit electrode assemblies are described in U.S. Pat. No. 8,715,203, Pub. No. US 2021/0202179, and Pub. No. US 2023/0043071.

### SUMMARY OF THE INVENTION

One aspect of the invention is directed to a first apparatus for applying an alternating electric field to a subject's body. The first apparatus comprises a first subassembly that has a front and a rear. The first subassembly includes a first layer of conductive material, at least one electrode element, at least one intermediate layer of material, and a plurality of conductive terminals. The first layer of conductive material is positioned at the front of the first subassembly in a central region of the first subassembly, so that a front surface of the first layer of conductive material serves as a front surface of the first subassembly in the central region of the first subassembly. The at least one electrode element is positioned between the first layer of conductive material and the rear of the first subassembly. Each of the electrode elements has a front surface. The at least one intermediate layer of material is positioned between the front surface of each of the electrode elements and a rear surface of the first layer of conductive material. The at least one intermediate layer of material is configured to either (a) capacitively couple each of the electrode elements to the first layer of conductive material or (b) conductively couple each of the electrode elements to the first layer of conductive material. The plurality of conductive terminals are positioned at the front of the first subassembly in a peripheral region of the first subassembly. The central region of the first subassembly has a perimeter, and the peripheral region of the first subassembly lies outside the perimeter. And the first layer of conductive material has an area of at least 10 cm².

Some embodiments of the first apparatus further comprise a second subassembly that has a front and a rear. The second subassembly includes a second layer of conductive material and a third layer of conductive material. The second layer of conductive material is positioned at the rear of the second subassembly, so that a rear surface of the second layer of conductive material serves as a rear surface of the second subassembly. The second layer of conductive material is shaped and dimensioned to overlap both the central region of the first subassembly and the peripheral region of the first subassembly. The third layer of conductive material is positioned at the front of the second subassembly in electrical contact with the second layer of conductive material, and the third layer of conductive material is configured to adhere to skin. The front surface of the first layer of conductive material is positioned against the rear surface of the second layer of conductive material when the first subassembly is in contact with the second subassembly. And the plurality of conductive terminals are positioned against a peripheral region of the second layer of conductive material when the first subassembly is in contact with and aligned with the second subassembly. Optionally, in these embodiments, at least one of the second layer of conductive material and the third layer of conductive material is a layer of conductive adhesive or conductive gel.

In some embodiments of the first apparatus, the at least one intermediate layer of material comprises (a) a layer of insulating material with a dielectric constant of at least 10 disposed on the front surface of each of the electrode elements, and (b) a first layer of conductive adhesive or conductive gel disposed on, and positioned in front of, the layer of insulating material. In these embodiments, the first layer of conductive material is a layer of conductive polymer. And the first layer of conductive material is disposed on, and positioned in front of, the first layer of conductive adhesive or conductive gel. Optionally, in these embodiments, the layer of conductive polymer comprises conductive silicone rubber.

In some embodiments of the first apparatus, the at least one intermediate layer of material comprises (a) a layer of insulating material with a dielectric constant of at least 10 disposed on the front surface of each of the electrode elements, and (b) a first layer of conductive adhesive or conductive gel disposed on, and positioned in front of, the layer of insulating material. In these embodiments, the first layer of conductive material is a layer of conductive polymer. And the first layer of conductive material is disposed on, and positioned in front of, the first layer of conductive adhesive or conductive gel. These embodiments further comprise a second subassembly that has a front and a rear. The second subassembly includes a second layer of conductive adhesive or gel, a layer of graphite, and a third layer of conductive material. The second layer of conductive adhesive or conductive gel is positioned at the rear of the second subassembly, so that a rear surface of the second layer of conductive adhesive or conductive gel serves as a rear surface of the second subassembly. The second layer of conductive adhesive or conductive gel is shaped and dimensioned to overlap both the central region of the first subassembly and the peripheral region of the first subassembly. The layer of graphite is disposed on, and positioned in front of, the second layer of conductive adhesive or conductive gel. The third layer of conductive material is disposed on, and positioned in front of, the layer of graphite, and the third layer of conductive material is configured to adhere to skin. In these embodiments, the front surface of the first layer of conductive material is positioned against the rear surface of the second layer of conductive adhesive or conductive gel when the first subassembly is in contact with the second subassembly. And the plurality of conductive terminals are positioned against a peripheral region of the second layer of conductive adhesive or conductive gel when the first subassembly is in contact with and aligned with the second subassembly.

Optionally, in the embodiments described in the previous paragraph, the third layer of conductive material comprises a conductive adhesive or a conductive gel. Optionally, in these embodiments, the layer of conductive polymer comprises conductive silicone rubber.

In some embodiments of the first apparatus, the at least one intermediate layer of material comprises a first layer of conductive adhesive or conductive gel disposed on, and positioned in front of, the front surface of each of the electrode elements. In these embodiments, the first layer of conductive material is disposed on, and positioned in front of, the first layer of conductive adhesive or conductive gel. And the first layer of conductive material is a layer of conductive silicone rubber.

In some embodiments of the first apparatus, the at least one intermediate layer of material comprises a first layer of conductive adhesive or conductive gel disposed on, and positioned in front of, the front surface of each of the electrode elements. In these embodiments, the first layer of conductive material is disposed on, and positioned in front of, the first layer of conductive adhesive or conductive gel. And the first layer of conductive material is a layer of conductive silicone rubber. These embodiments further comprise a second subassembly that has a front and a rear. The second subassembly includes a second layer of conductive adhesive or conductive gel, a layer of graphite, and a third layer of conductive material. The second layer of conductive adhesive or conductive gel is positioned at the rear of the second subassembly, so that a rear surface of the second layer of conductive adhesive or conductive gel serves as a rear surface of the second subassembly. The second layer of conductive adhesive or conductive gel is shaped and dimensioned to overlap both the central region of the first subassembly and the peripheral region of the first subassembly. The layer of graphite is disposed on, and positioned in front of, the second layer of conductive adhesive or conductive gel. The third layer of conductive material is disposed on, and positioned in front of, the layer of graphite, and the third layer of conductive material is configured to adhere to skin. The front surface of the first layer of conductive material is positioned against the rear surface of the second layer of conductive adhesive or conductive gel when the first subassembly is in contact with the second subassembly. And the plurality of conductive terminals are positioned against a peripheral region of the second layer of conductive adhesive or conductive gel when the first subassembly is in contact with and aligned with the second subassembly.

In some embodiments of the first apparatus, the at least one intermediate layer of material comprises: (a) a layer of insulating material with a dielectric constant of at least 10 disposed on the front surface of each of the electrode elements, (b) a first layer of conductive adhesive or conductive gel disposed on, and positioned in front of, the layer of insulating material, and (c) a layer of graphite disposed on, and positioned in front of, the first layer of conductive adhesive or conductive gel. In these embodiments, the first layer of conductive material comprises a conductive adhesive or conductive gel. And the first layer of conductive material is disposed on, and positioned in front of, the layer of graphite.

Optionally, the embodiments described in the previous paragraph may further comprise a second subassembly that has a front and a rear. The second subassembly includes a layer of conductive polymer and a third layer of conductive material. The layer of conductive polymer is positioned at the rear of the second subassembly, so that a rear surface of the layer of conductive polymer serves as a rear surface of the second subassembly. The layer of conductive polymer is shaped and dimensioned to overlap both the central region of the first subassembly and the peripheral region of the first subassembly. The third layer of conductive material is disposed on, and positioned in front of, the layer of conductive polymer. And the third layer of conductive material is configured to adhere to skin. In these embodiments, the front surface of the first layer of conductive material is positioned against the rear surface of the layer of conductive polymer when the first subassembly is in contact with the second subassembly. And the plurality of conductive terminals are positioned against a peripheral region of the layer of conductive polymer when the first subassembly is in contact with and aligned with the second subassembly. Optionally, in these embodiments, the layer of conductive polymer comprises conductive silicone rubber.

In some embodiments of the first apparatus, each of the conductive terminals comprises a conductive pad of a printed circuit.

In some embodiments of the first apparatus, the plurality of conductive terminals comprises at least four conductive terminals, and the apparatus further comprises a circuit configured to measure resistances or electrical continuities between a plurality of pairs of the at least four conductive terminals.

Optionally, in the embodiments described in the previous paragraph, the circuit can be further configured to generate an output when the measured resistances or electrical continuities deviate from values that would be expected when all of the conductive terminals are making good contact with a sheet of conductive material.

Optionally, in the embodiments described in the previous paragraph, the circuit can be further configured to disable operation of the first subassembly in response to the generation of the output. Optionally, in these embodiments, the at least four conductive terminals comprises a first conductive terminal positioned near a first corner of the first subassembly, a second conductive terminal positioned near a second corner of the first subassembly, a third conductive terminal positioned near a third corner of the first subassembly, and a fourth conductive terminal positioned near a fourth corner of the first subassembly.

In some embodiments of the first apparatus, the plurality of conductive terminals comprises at least three conductive terminals, and the apparatus further comprises a circuit configured to measure resistances or electrical continuities between a plurality of pairs of the at least three conductive terminals.

Another aspect of the invention is directed to a second apparatus for applying an alternating electric field to a subject's body. The second apparatus comprises a first subassembly that has a front and a rear. The first subassembly includes a first layer of conductive material, at least one electrode element, at least one intermediate layer of material, a plurality of light sources, and a plurality of light detectors. The first layer of conductive material is positioned at the front of the first subassembly in a central region of the first subassembly, so that a front surface of the first layer of conductive material serves as a front surface of the first subassembly in the central region of the first subassembly. The at least one electrode element is positioned between the first layer of conductive material and the rear of the first subassembly, and each of the electrode elements has a front surface. The at least one intermediate layer of material is positioned between the front surface of each of the electrode elements and a rear surface of the first layer of conductive material, and the at least one intermediate layer of material is configured to either (a) capacitively couple each of the electrode elements to the first layer of conductive material or (b) conductively couple each of the electrode elements to the first layer of conductive material. The plurality of light sources are positioned in a peripheral region of the first subassembly, and each of the plurality of light sources is aimed in a forward direction. The plurality of light detectors are positioned in the peripheral region of the first subassembly, and each of the plurality of light detectors is positioned to detect whether light emanating from a respective one of the plurality of light sources is being reflected back by a respective portion of a second subassembly. The central region of the first subassembly has a perimeter, and the peripheral region of the first subassembly lies outside the perimeter.

In some embodiments of the second apparatus, the first layer of conductive material has an area of at least 10 cm².

Some embodiments of the second apparatus further comprise a second subassembly that has a front and a rear. The second subassembly includes a second layer of conductive material and a third layer of conductive material. The second layer of conductive material is positioned at the rear of the second subassembly, so that a rear surface of the second layer of conductive material serves as a rear surface of the second subassembly, and the second layer of conductive material is shaped and dimensioned to overlap both the central region of the first subassembly and the peripheral region of the first subassembly. The third layer of conductive material is positioned at the front of the second subassembly in electrical contact with the second layer of conductive material, and the third layer of conductive material is configured to adhere to skin. In these embodiments, the front surface of the first layer of conductive material is positioned against the rear surface of the second layer of conductive material when the first subassembly is in contact with the second subassembly. And the plurality of light sources and the plurality of light detectors are positioned against a peripheral region of the second layer of conductive material when the first subassembly is in contact with and aligned with the second subassembly. Optionally, in these embodiments, at least one of the second layer of conductive material and the third layer of conductive material is a layer of conductive adhesive or conductive gel.

Another aspect of the invention is directed to a first method of inhibiting or preventing the operation of a system for applying alternating electric fields to a subject's body. The system includes a second subassembly that is configured for positioning on the subject's body and a first subassembly that is removably affixed to the second subassembly. The first method comprises applying alternating electric fields to the subject's body via the first subassembly and the second subassembly when the first subassembly is affixed to the second subassembly; determining whether the first subassembly and the second subassembly are making good contact with each other; and stopping the applying of the alternating electric fields to the subject's body when a determination has been made that the first subassembly and the second subassembly are not making good contact with each other.

Some instances of the first method further comprise providing at least one of a visual signal, an auditory signal, and a haptic signal to indicate that the first subassembly and the second subassembly are not making good contact with each other.

In some instances of the first method, the determining comprises measuring at least one resistance or electrical continuity between a plurality of conductive terminals positioned on the first subassembly. In these instances, each of the resistance or electrical continuity measurements travels through a component in the second subassembly.

In some instances of the first method, the determining comprises illuminating a plurality of light sources positioned on the first subassembly, wherein each of the plurality of light sources is aimed towards the second subassembly; and detecting, using a plurality of light detectors positioned on the first subassembly, how much light from the plurality of light sources has arrived at the plurality of light detectors.

Another aspect of the invention is directed to a second method of inhibiting or preventing the operation of a system for applying alternating electric fields to a subject's body. The system includes a second subassembly that is configured for positioning on the subject's body and a first subassembly that is removably affixed to the second subassembly. The second method comprises applying alternating electric fields to the subject's body via the first subassembly and the second subassembly when the first subassembly is affixed to the second subassembly; determining whether the first subassembly and the second subassembly are making good contact with each other and aligned with each other; and stopping the applying of the alternating electric fields to the subject's body when a determination has been made that the first subassembly and the second subassembly are either not making good contact with each other or not aligned with each other.

Some instances of the second method further comprise providing at least one of a visual signal, an auditory signal, and a haptic signal to indicate that the first subassembly and the second subassembly are either not making good contact with each other or not aligned with each other.

In some instances of the second method, the determining comprises measuring at least one resistance or electrical continuity between a plurality of conductive terminals positioned on the first subassembly. In these instances, each of the resistance or electrical continuity measurements travels through a component in the second subassembly. And the plurality of conductive terminals are positioned on the first subassembly so that electrical continuity is interrupted when the first subassembly and the second subassembly are not aligned with each other.

In some instances of the second method, the determining comprises illuminating a plurality of light sources positioned on the first subassembly, wherein each of the plurality of light sources is aimed towards the second subassembly; and detecting, using a plurality of light detectors positioned on the first subassembly, how much light from the plurality of light sources has arrived at the plurality of light detectors.

### BRIEF DESCRIPTION OF THE DRAWINGS

FIG. 1 is a plan view of a first-type electrode assembly (e.g., a transducer array) for applying alternating electric fields to a subject's body.

FIG. 2 is a section view of the FIG. 1 electrode assembly.

FIG. 3 is a schematic representation of two FIG. 1 electrode assemblies positioned on a subject's body, and a system for driving those electrode assemblies.

FIG. 4 is a flowchart that the controller implements to switch an AC signal to the electrode assemblies in the FIG. 3 embodiment on or off.

FIG. 5 is a plan view of a second-type electrode assembly (e.g., a transducer array) for applying alternating electric fields to a subject's body.

FIG. 6 is a section view of the FIG. 5 electrode assembly.

FIG. 7 is a plan view of a third-type electrode assembly (e.g., a transducer array) for applying alternating electric fields to a subject's body.

FIG. 8 is a section view of the FIG. 7 electrode assembly.

FIG. 9 depicts a spatial relationship between two subassemblies that are not aligned with each other.

FIG. 10 is a flowchart that the controller implements to determine that the two subassemblies have either separated from each other or are not aligned.

Various embodiments are described in detail below with reference to the accompanying drawings, wherein like reference numerals represent like elements.

### DESCRIPTION OF THE PREFERRED EMBODIMENTS

As noted above, conventional electrode assemblies for applying TTFields to a subject's body each include a plurality of parts that are all incorporated into a single integrated unit. Alternating electric fields can also be applied to a subject's body using two or more electrode assemblies, each of which is divided into two discrete subassemblies that are removably connectable to each other. But when an electrode assembly is divided into two discrete subassemblies that are removably connectable, it can be important for safety reasons that those two discrete subassemblies remain in intimate contact with each other during use. It can also be important to ensure that the two discrete subassemblies are aligned with each other both prior to and during use.

Section 1 of this application describes a number of electrode assemblies that are divided into two discrete subassemblies, and it also describes a variety of approaches for determining whether those two discrete subassemblies are, in fact, in intimate contact. As long as the two discrete subassemblies remain in intimate contact, the system applies an AC voltage to the electrode assemblies. But if it is determined that the subassemblies within any given electrode assembly are no longer in intimate contact, the system will turn off the AC voltage to that electrode assembly.

Section 2 of this application describes how the same electrode assemblies described in Section 1 can also be used for determining whether the two discrete subassemblies are properly aligned with each other. If the two subassemblies are not properly aligned prior to use, the system will not activate that electrode assembly. And if the two subassemblies shift to an improper alignment during use, the system will deactivate that electrode assembly. If a problem is detected, the system informs the user of the problem.

### Section 1—Intimate Contact

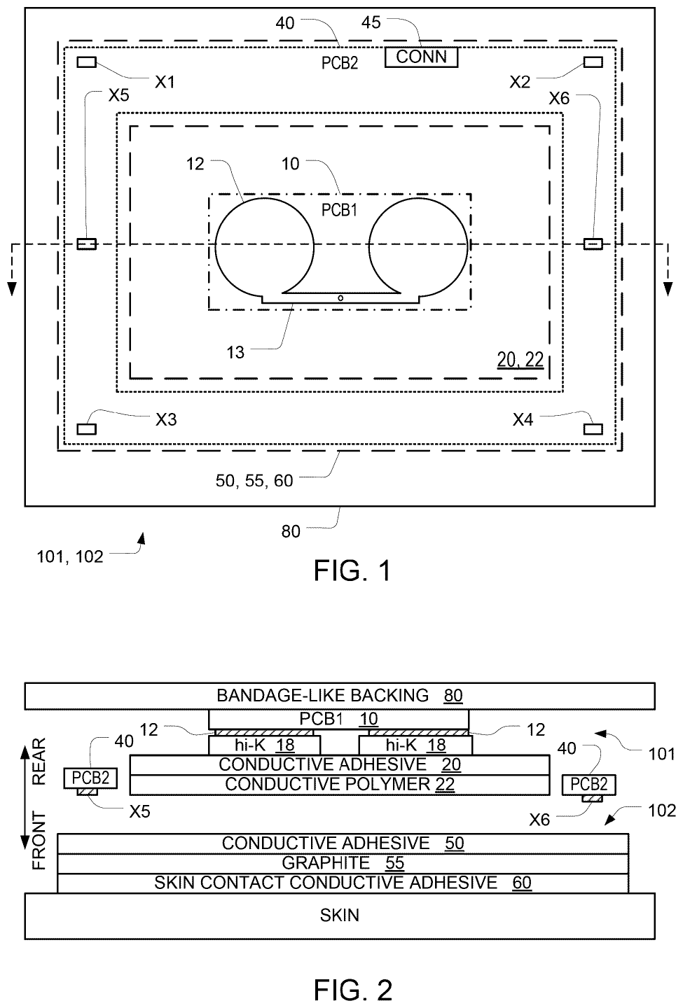

FIG. 1 is a plan view of an electrode assembly (e.g., a transducer array) for applying alternating electric fields (e.g., TTFields) to a subject's body that is divided into a first (i.e., rear) subassembly 101 (electrode subassembly) and a second (i.e., front) subassembly 102 (skin-contact subassembly), and FIG. 2 is a section view of the same electrode assembly. During use, the first and second subassemblies 101, 102 will be pressed against each other and in intimate contact. But those two subassemblies 101, 102 are shown spaced apart in FIG. 2 so that the boundary between the two subassemblies will be clear.

Referring to FIGS. 1-2, the first subassembly 101 includes a first layer of conductive material, which is a layer of conductive polymer 22 in the FIGS. 1-2 example. The layer of conductive polymer 22 is positioned at the front of the first subassembly in a central region of the first subassembly, so that a front surface of the layer of conductive polymer 22 serves as a front surface of the first subassembly 101 in the central region of the first subassembly. Thus, in this example, the perimeter of the front surface of the layer of conductive polymer 22 is the perimeter of the central region of the first subassembly. The layer of conductive polymer 22 has an area of at least 10 cm². Examples of suitable materials for the layer of conductive polymer 22 include conductive versions of polymers including, but not limited to silicone, silicone rubber, natural rubber, poly cis-isoprene, polyisobutylene, chloroprene, cis-polybutadiene, styrene-butadiene, styrene-acrylonitrile-butadiene, polyurethane, EPDM, EVA polymers, and perfluoropolymers. Such polymers can be modified to produce conductive versions of the polymers by infusing them with conductive particles, such as, for example, metal particles or carbon particles. Carbon particles may include, for example, carbon flakes, carbon granules, carbon fibers, carbon black powder, graphite powder, carbon nanotubes, carbon nanowires, and the like.

The first subassembly 101 also includes at least one electrode element 12 positioned between the layer of conductive polymer 22 and the rear of the first subassembly 101. Each of the electrode elements 12 has a front surface. In the example depicted in FIGS. 1-2, the electrode elements 12 are metal (e.g. copper) pads that are disposed on the front face of a first PCB 10. When more than one metal pad 12 is included (as depicted in FIGS. 1-2), all the metal pads 12 can be connected by metal traces 13. Note that as used herein, the term “PCB” refers to a printed circuit board, and this term encompasses rigid PCBs (e.g., with copper traces on a rigid epoxy board), flex circuits (e.g., with copper traces on a flexible polyimide substrate), and printed circuits made by printing a conductive ink on a flexible substrate.

At least one intermediate layer of material is positioned between the front surface of each of the electrode elements 12 and a rear surface of the first layer of conductive material (i.e., the layer of conductive polymer 22 in the FIGS. 1-2 example).

In the example depicted in FIGS. 1-2, the at least one intermediate layer of material comprises (a) a layer of insulating material 18 (labelled “hi-K” in FIGS. 2, 6, 8) with a dielectric constant of at least 10 disposed on the front surface of each of the electrode elements 12, and (b) a first layer of conductive adhesive 20 disposed on, and positioned in front of, the layer of insulating material 18. The layer of conductive polymer 22 is disposed on, and positioned in front of, the first layer of conductive adhesive 20. And because the layer of insulating material 18 has a high dielectric constant, the layer of insulating material 18 and the first layer of conductive adhesive 20 will collectively capacitively couple each of the electrode elements 12 to the layer of conductive polymer 22.

Examples of suitable materials for the first layer of conductive adhesive 20 include, but are not limited to, the OMNI-WAVE™ adhesive compositions manufactured and sold by FLEXcon® (Spencer, MA, USA), such as the developmental product FLX068983-FLEXcon® OMNI-WAVE™ TT 200 BLACK H-502 150 POLY H-9 44PP-8; and the adhesives from ADHESIVE RESEARCH, such as ARcare® 8006 electrically conductive adhesive composition manufactured and sold by Adhesives Research, Inc. (Glen Rock, PA, USA). Alternatively, Electrically Conductive Adhesive Transfer Tape 9712 or Electrically Conductive Adhesive Transfer Tape 9713 (both manufactured by 3M, Saint Paul, MN, USA) may also be used. Note that the first layer of conductive adhesive 20 depicted in the FIG. 1-2 example may be replaced with a layer of conductive gel (e.g. a conductive hydrogel).

Examples of suitable materials for the layer of insulating material 18 include, but are not limited to, at least one of Poly (VDF-TrFE-CTFE), Poly (VDF-TrFE-CFE), and Poly (VDF-TrFE-CFE-CTFE), and/or ceramic nanoparticles mixed into at least one of Poly (VDF-TrFE), P (VDF-HFP), PVDF.

In an alternative embodiment (not shown) the layer of insulating material 18 is omitted, in which case the at least one intermediate layer of material will comprise just the first layer of conductive adhesive 20, which is disposed on, and positioned in front of, the electrode elements 12. In this embodiment, the layer of conductive polymer 22 is disposed on, and positioned in front of, the first layer of conductive adhesive 20. And because the layer of insulating material 18 is not present, the at least one intermediate layer of material (i.e., the first layer of conductive adhesive 20) will conductively couple each of the electrode elements 12 to the layer of conductive polymer 22.

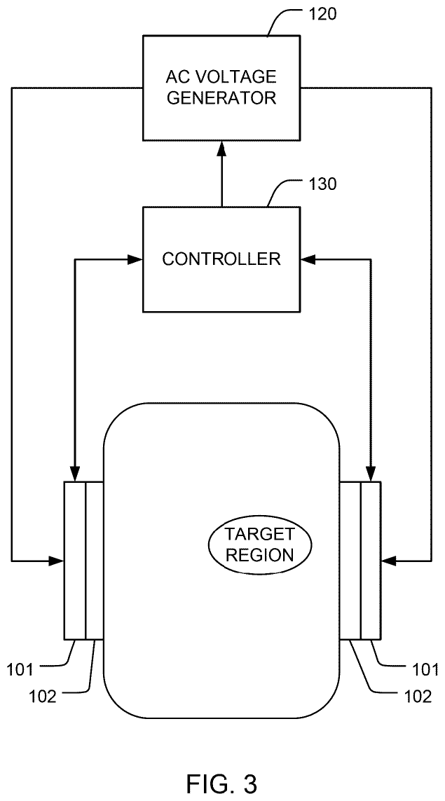

The first subassembly 101 also includes a plurality of conductive terminals X1-X6 positioned at the front of the first subassembly in a peripheral region of the first subassembly. And conductive traces or wires (not shown) are provided between each of the conductive terminals X1-X6 and a respective pin of the connector 45 to provide the controller 130 (FIG. 3) with access to the conductive terminals X1-X6. The peripheral region of the first subassembly lies outside the perimeter of the central region of the first subassembly. In the example depicted in FIGS. 1-2, the first subassembly 101 is rectangular, each of the conductive terminals X1-X6 comprises a conductive pad of a second PCB 40, and conductive terminals X1, X2, X3, and X4 are respectively positioned near each of the four corners of the first subassembly 101. In alternative embodiments (not shown) in which the first subassembly 101 is not rectangular, the first subassembly may not have corners. In this situation, the conductive pads X1-X6 should preferably be positioned near the edges of the first subassembly 101 (e.g., within 1 cm of the edges, within 5 mm of the edges, or within 3 mm of the edges). Note that while the conductive terminals X1-X6 are located on a different PCBs (PCB 2/PCB 40) than the electrode elements 12 in the embodiment depicted in FIGS. 1-2 (PCB 1/PCB 10), in alternative embodiments (not shown) the conductive terminals X1-X6 and the electrode elements 12 could all be located on a single PCB.

The second subassembly 102 includes a second layer of conductive material 50, (which is a second layer of conductive adhesive 50 in the FIGS. 1-2 example) positioned at the rear of the second subassembly, so that a rear surface of the second layer of conductive adhesive 50 serves as a rear surface of the second subassembly 102. The second layer of conductive adhesive 50 is shaped and dimensioned to overlap both the central region of the first subassembly (i.e., the region that corresponds to the layer of conductive polymer 22) and the peripheral region of the first subassembly (i.e., the region that corresponds to the second PCB 40). A layer of graphite 55 is disposed on, and positioned in front of, the first layer of conductive adhesive 50. And a third layer of conductive material, 60 (which is a conductive adhesive in the FIGS. 1-2 example) is disposed on, and positioned in front of, the layer of graphite 55. The third layer of conductive material 60 is configured to adhere to skin.

Examples of suitable materials for the second layer of conductive adhesive 50 include, but are not limited to, the same materials described above for the first layer of conductive adhesive 20. Examples of suitable materials for the layer of graphite 55 include, but are not limited to, synthetic graphite, pyrolytic graphite (including, but not limited to, Pyrolytic Graphite Sheet (PGS), available from Panasonic Industry, Kadoma, Osaka, Japan), graphitized polymer film (e.g., graphitized polyimide film, including, but not limited to, that supplied by Kaneka Corp., Moka, Tochigi, Japan), or graphite foil made from compressed high purity exfoliated mineral graphite (including, but not limited to, that supplied by MinGraph® 2010A Flexible Graphite, available from Mineral Seal Corp., Tucson, Arizona, USA). In alternative embodiments, instead of using a layer of graphite 55, a layer of another anisotropic material may be used.

Examples of suitable materials for the third layer of conductive material 60 (which is a conductive adhesive in the FIGS. 1-2 example) include any suitable biocompatible adhesive that is designed to be removably affixed to a person's skin. These may also include, but are not limited to, the same materials described above for the first layer of conductive adhesive 20. Note that the second layer of conductive adhesive 50 and/or the third layer of conductive material 60 depicted in the FIG. 1-2 example may be replaced with a layer of conductive gel (e.g. a conductive hydrogel).

A flexible backing 80 (e.g., a bandage-like backing) is positioned behind the first and second PCBs 10, 40, and this flexible backing 80 is configured to support both of those PCBs. At least a portion of the flexible backing 80 extends laterally beyond the second PCB 40, and the front of this portion is covered with a biocompatible adhesive that adheres to skin. This portion of the flexible backing 80 helps hold the first and second subassemblies 101, 102 against the subject's skin.

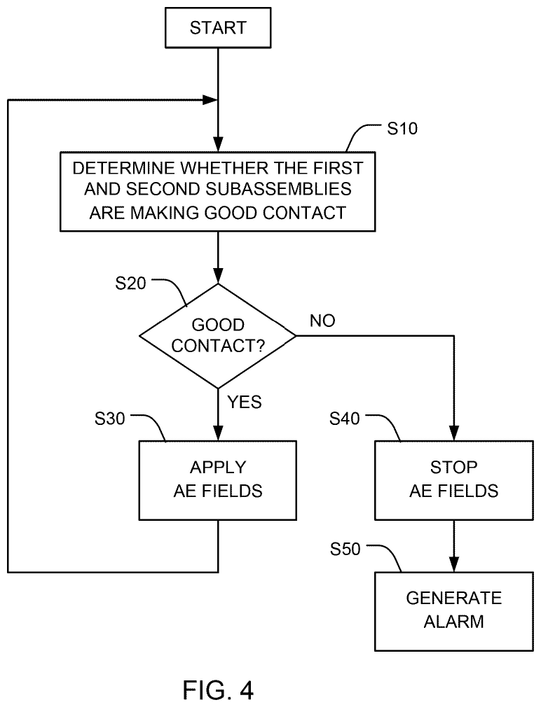

When the first subassembly 101 is in contact with the second subassembly 102 (i.e., by moving those two subassemblies towards each other until the gap between those two subassemblies depicted in FIG. 2 is eliminated), the front surface of the layer of conductive polymer 22 will be positioned against the rear surface of the second layer of conductive adhesive 50, and the plurality of conductive terminals X1-X6 will be positioned against a peripheral region of the second layer of conductive adhesive 50. This is important for three reasons. First, due to the adhesive nature of the conductive adhesive 50, the first and second subassemblies 101, 102 will adhere to each other (until such time when they are pulled apart). Second, the contact between the layer of conductive polymer 22 (in the first subassembly 101) and the second layer of conductive adhesive 50 (in the second subassembly 102) allows an AC signal to traverse those two components, as described below in connection with FIGS. 3-4. And third, the contact between the conductive terminals X1-X6 (in the first subassembly 101) and the second layer of conductive adhesive 50 (in the second subassembly 102) allows the system to determine whether the first and second subassemblies 101, 102 remain in intimate contact, as described below in connection with FIGS. 3-4.

FIGS. 3 and 4 depict one example of how to use the electrode assemblies 101/102 depicted in FIGS. 1-2 to apply alternating electric fields (e.g., TTFields) to a target region in a subject's body, and how the AC signal to those electrode assemblies can be stopped if the first and second subassemblies within any given electrode assembly begin to separate. More specifically, FIG. 3 is a schematic representation of two electrode assemblies 101/102 positioned on a subject's body, and a system for driving those electrode assemblies. And FIG. 4 is a flowchart that the controller 130 (FIG. 3) implements to either continue or stop the AC signal to the electrode assemblies 101/102.

Assuming that the first and second subassemblies 101, 102 have been previously adhered to each other to form an electrode assembly, one electrode assembly 101/102 is positioned on the subject's skin on one side of the target region, and a second identical electrode assembly 101/102 is positioned on the subject's skin on the opposite side of the target region. Each of the electrode assemblies 101/102 is self adhesive (due to the nature of the frontmost layer 60 and the flexible backing 80), and will therefore stick to the subject's skin.

To impose alternating electric fields in the target region, the AC voltage generator 120 applies an AC voltage (e.g., between 50 kHz and 5 MHz, or 75-300 kHz) between the metal pads 12 (FIG. 2) of the first electrode assembly 101/102 and the metal pads 12 in the second electrode assembly 101/102. The AC signal from the AC voltage generator 120 arrives at each of the first and second electrode assemblies 101/102 via a set of cables that route the AC signals (e.g., via the metal traces 13 and other intervening components, not shown) to the metal pads 12 of the first subassembly 101 of each electrode assembly 101/102.

Because the first and second subassemblies 101, 102 within each electrode assembly are adhered to each other, an AC signal that is applied to the metal pads 12 of opposing electrode assemblies 101/102 will be capacitively coupled across the layer of insulating material 18 in each of the electrode assemblies, and will subsequently pass through all of the conductive layers in front of the layer of insulating material 18 (i.e., the first layer of conductive adhesive 20, the layer of conductive polymer 22, the second layer of conductive adhesive 50, the layer of graphite 55, and the third layer of conductive material 60 (which is a conductive adhesive in the FIGS. 1-2 example). Notably, the layer of graphite 55 is both thermally conductive and electrically conductive, and it acts to spread both the flow of current and heat in all four directions (i.e., to the right, to the left, into the page, and out of the page in FIG. 2, which corresponds to right, left, up, and down in FIG. 1). And because the third layer of conductive material 60 of each of the opposing electrode assemblies 101/102 is in contact with the subject's skin on opposite sides of the target region, alternating electric fields will be induced through the target region.

In addition, because the first and second subassemblies 101, 102 within each electrode assembly are adhered to each other, the controller 130 can determine whether the first subassembly and the second subassembly are making good contact with each other. This can be accomplished, for example, using a circuit that sends electrical signals into the first subassembly 101 (e.g., via the connector 45) to measure the resistance of a path between respective pairs of the conductive terminals (e.g., X1 and X2, X3 and X4, etc.). For when the first and second subassemblies 101, 102 are in fact adhered to each other, all of the conductive terminals X1-X6 will be in contact with the second layer of conductive adhesive 50 in the FIGS. 1-2 example, in which case the conductive adhesive 50 will provide a low resistance path between any given pair of those terminals (e.g., X1 and X2, X3 and X4, etc.). On the other hand, if the first and second subassemblies 101, 102 become separated to the point where one or more of the conductive terminals X1-X6 is no longer in intimate contact with the second layer of conductive adhesive 50, the resistance of the path between some (or all) of those terminals will rise above the value that would be expected when the terminals are all making good contact with the second layer of conductive adhesive 50.

Because the controller 130 can determine whether the first and second subassemblies 101, 102 are making good contact with each other (e.g., as described above or using an alternative approach, including but not limited to measuring electrical conductances or continuities instead of measuring resistances), the controller 130 can selectively inhibit or prevent the application of alternating electric fields to the subject's body if the first and second subassemblies 101, 102 are no longer making good contact with each other. One example of how the controller 130 can accomplish this is by implementing the method depicted in FIG. 4.

More specifically, in step S10, the controller determines whether the first and second subassemblies 101, 102 are making good contact with each other (e.g., as described above). Step S20 is a branching step. When a determination has been made in S10 that the first and second subassemblies 101, 102 are making good contact with each other, the processing flow proceeds to S30, where alternating electric fields (“AE Fields” in FIG. 4) are applied to the subject's body (via the first and second subassemblies 101, 102). On the other hand, when a determination has been made in S10 that the first and second subassemblies 101, 102 are not making good contact with each other, the processing flow proceeds to S40, where the application of the alternating electric fields is stopped. Optionally, after stopping the alternating electric fields, an alarm or alert (e.g., a visual signal, an auditory signal, or a haptic signal) can be generated in S50 to notify the user that the first and second subassemblies 101, 102 are no longer making good contact with each other. The user can then take appropriate corrective action (e.g., pressing the first and second subassemblies 101, 102 together, or replacing one or both of those subassemblies).

Advantageously, stopping the AC signal that is applied to the electrode assemblies 101/102 when the first and second subassemblies 101, 102 included therein are no longer making good contact with each other avoids the safety problems that can arise when those subassemblies begin to separate from each other.

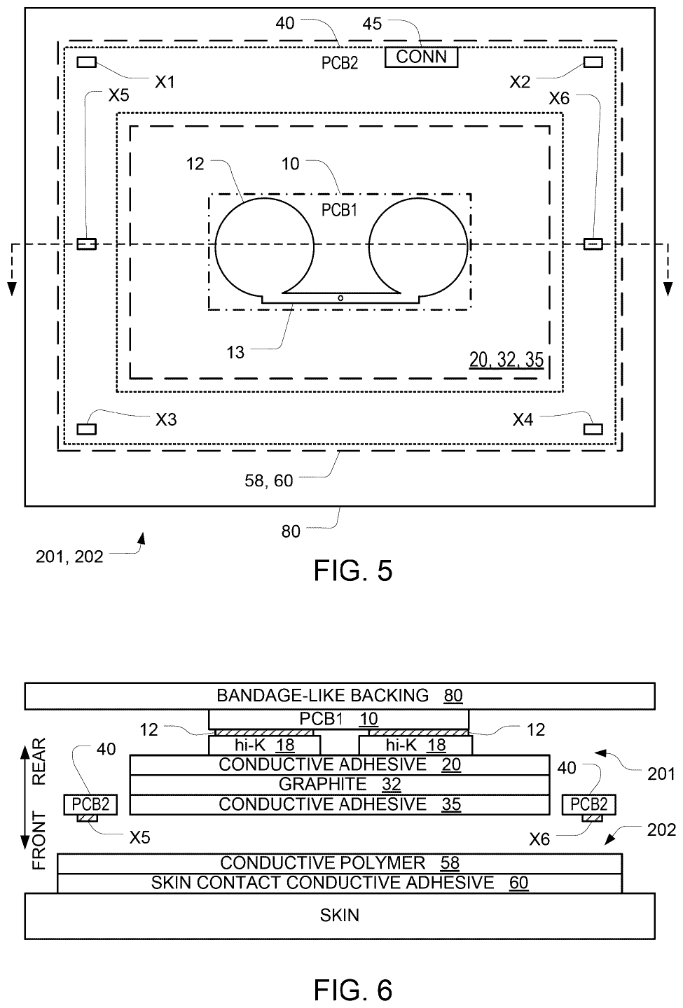

Notably, the specific components described above in connection with FIG. 1-2 and the distribution of those components within the first and second subassemblies described above is not the only way to form an electrode assembly that is divided into two discrete subassemblies that are removably connectable. For example, one different approach for dividing an electrode assembly into two subassemblies is described below in connection with FIGS. 5-6, and a variety of other different approaches can also be implemented.

FIG. 5 is a plan view of a different electrode assembly (e.g., a transducer array) for applying alternating electric fields (e.g., TTFields) to a subject's body that is divided into a first (i.e., rear) subassembly 201 (electrode subassembly) and a second (i.e., front) subassembly 202 (skin-contact subassembly), and FIG. 6 is a section view of the same electrode assembly. During use, the first and second subassemblies 201, 202 will be pressed against each other and in intimate contact. But those two subassemblies 201, 202 are shown spaced apart in FIG. 6 so that the boundary between the two subassemblies will be clear.

Referring to FIGS. 5-6, the first subassembly 201 includes a first layer of conductive material 35, which is a layer of conductive adhesive 35 in the FIGS. 5-6 example. The first layer of conductive material 35 is positioned at the front of the first subassembly in a central region of the first subassembly, so that a front surface of the first layer of conductive material 35 serves as a front surface of the first subassembly 201 in the central region of the first subassembly. Thus, in this example, the perimeter of the front surface of the first layer of conductive material 35 is the perimeter of the central region of the first subassembly. The first layer of conductive material 35 has an area of at least 10 cm². The first subassembly 201 also includes at least one electrode element 12 positioned between the first layer of conductive material 35 and the rear of the first subassembly 201. Each of the electrode elements 12 has a front surface. In the example depicted in FIGS. 5-6, the electrode elements 12 are metal (e.g., copper) pads that are disposed on a first PCB 10. When more than one metal pad 12 is included (as depicted in FIGS. 5-6), all the metal pads 12 can be connected by metal traces 13.

At least one intermediate layer of material is positioned between the front surface of each of the electrode elements 12 and a rear surface of the first layer of conductive material 35.

In the example depicted in FIGS. 5-6, the at least one intermediate layer of material comprises (a) a layer of insulating material 18 (labelled “hi-K” in FIG. 6) with a dielectric constant of at least 10 disposed on the front surface of each of the electrode elements 12, (b) a first layer of conductive adhesive 20 disposed on, and positioned in front of, the layer of insulating material 18, and (c) a layer of graphite 32 disposed on, and positioned in front of, the first layer of conductive adhesive 20. The first layer of conductive material 35 is disposed on, and positioned in front of, the layer of graphite 32. Because the layer of insulating material 18 has a high dielectric constant, the layer of insulating material 18, the first layer of conductive adhesive 20 and the layer of graphite 32 will collectively capacitively couple each of the electrode elements 12 to the first layer of conductive material 35.

Examples of suitable materials for the layer of insulating material 18 and the first layer of conductive adhesive 20 are the same as those described above in connection with the FIGS. 1-2 embodiment. Note that the first layer of conductive adhesive 20 depicted in the FIG. 5-6 example may be replaced with a layer of conductive gel (e.g., a conductive hydrogel). Examples of suitable materials for the layer of graphite 32 are the same as those described above for the layer of graphite 55 in the FIGS. 1-2 embodiment. And examples of suitable materials for the first layer of conductive material 35 are the same as those described above for the first layer of conductive adhesive 20 in the FIGS. 1-2 embodiment, as well as conductive gels (e.g., conductive hydrogels).

In an alternative embodiment (not shown) the layer of insulating material 18 is omitted, in which case the at least one intermediate layer of material will comprise just the first layer of conductive adhesive 20 and the layer of graphite 32. In this embodiment, the first layer of conductive adhesive 20 is disposed on, and positioned in front of, the electrode elements 12. And the layer of graphite 32 is disposed on, and positioned in front of, the first layer of conductive adhesive 20. Because the layer of insulating material 18 is not present, the at least one intermediate layer of material (i.e., the first layer of conductive adhesive 20 and the layer of graphite 32) will conductively couple each of the electrode elements 12 to the first layer of conductive material 35.

The first subassembly 201 also includes a plurality of conductive terminals X1-X6 positioned at the front of the first subassembly in a peripheral region of the first subassembly. And conductive traces or wires (not shown) are provided between each of the conductive terminals X1-X6 and a respective pin of the connector 45 to provide the controller 130 (FIG. 3) with access to the conductive terminals X1-X6. The peripheral region of the first subassembly lies outside the perimeter of the central region of the first subassembly. In the example depicted in FIGS. 5-6, the first subassembly 201 is rectangular, each of the conductive terminals X1-X6 comprises a conductive pad of a second PCB 40, and conductive terminals X1, X2, X3, and X4 are respectively positioned near each of the four corners of the first subassembly 201. In alternative embodiments (not shown) in which the first subassembly 201 is not rectangular, the first subassembly may not have corners. In this situation, the conductive pads X1-X6 should preferably be positioned near the edges of the first subassembly 201 (e.g., within 1 cm of the edges, within 5 mm of the edges, or within 3 mm of the edges). Note that while the conductive terminals X1-X6 are located on a different PCBs (PCB 2/PCB 40) than the electrode elements 12 in the embodiment depicted in FIGS. 5-6 (PCB 1/PCB 10), in alternative embodiments (not shown) the conductive terminals X1-X6 and the electrode elements 12 could all be located on a single PCB.

The second subassembly 202 includes a second layer of conductive material, which is a layer of conductive polymer 58 in the FIGS. 5-6 example. The layer of conductive polymer 58 is positioned at the rear of the second subassembly, so that a rear surface of the layer of conductive polymer 58 serves as a rear surface of the second subassembly 202. The layer of conductive polymer 58 is shaped and dimensioned to overlap both the central region of the first subassembly (i.e., the region that corresponds to the first layer of conductive material 35) and the peripheral region of the first subassembly (i.e., the region that corresponds to the second PCB 40). The second subassembly 202 also includes a third layer of conductive material 60 (which is a conductive adhesive in the FIGS. 5-6 example) disposed on, and positioned in front of, the layer of conductive polymer 58, and the third layer of conductive material 60 is configured to adhere to skin. Examples of suitable materials for the layer of conductive polymer 58 are the same as those described above for the layer of conductive polymer 22 in the FIGS. 1-2 embodiment. And examples of suitable materials for the third layer of conductive material 60 are the same as those described above for the corresponding layer in the FIGS. 1-2 embodiment.

A flexible backing 80 is positioned behind the first and second PCBs 10, 40, and this flexible backing 80 is similar to the flexible backing described above in connection with FIGS. 1-2.

When the first subassembly 201 is in contact with the second subassembly 202 (i.e., by moving those two subassemblies towards each other until the gap between those two subassemblies depicted in FIG. 6 is eliminated), the front surface of the conductive material 35 will be positioned against the rear surface of the layer of conductive polymer material 58, and the plurality of conductive terminals X1-X6 will be positioned against a peripheral region of the layer of conductive polymer material 58. And due to the adhesive nature of the conductive material 35 (which is a layer of conductive adhesive 35 in the FIGS. 5-6 example), the first and second subassemblies 201, 202 will adhere to each other (until such time when they are pulled apart). This is important for reasons very similar to the three reasons described above in connection with the FIGS. 1-2 embodiment.

The way that the electrode assemblies 201/202 depicted in FIGS. 5-6 are used is similar to way the electrode assemblies 101/102 are used, as described above in connection with FIGS. 3-4. Indeed, the most significant difference between the electrode assemblies 201/202 and the electrode assemblies 101/102 (described above) is the set of conductive layers in front of the layer of insulating material 18, and the sequencing of those conductive layers. More specifically, in the FIGS. 5-6 embodiment, the following set of conductive layers, in the following sequence, is disposed in front of the layer of insulating material 18: the first layer of conductive adhesive 20, the layer of graphite 32, the first layer of conductive material 35, the layer of conductive polymer 58, and the third layer of conductive material 60 (which is a conductive adhesive in the FIGS. 5-6 example). Once again, when used as described in FIG. 3 to treat a target region in a subject's body, because the third layer of conductive material 60 of each of the opposing electrode assemblies 201/202 is in contact with the subject's skin on opposite sides of the target region, alternating electric fields will be induced through the target region.

The same resistance-based approach described above for determining whether the first and second subassemblies 101, 102 are making good contact with each other can be used in the context of the first and second subassemblies 201, 202 depicted in FIGS. 5-6, as well as a variety of alternative approaches including those described above in connection with FIG. 3). And the same approach described above in connection with FIG. 4 can also be used to stop the application of alternating electric fields when the electrode assemblies 201/202 are used, as well as a variety of alternative approaches.

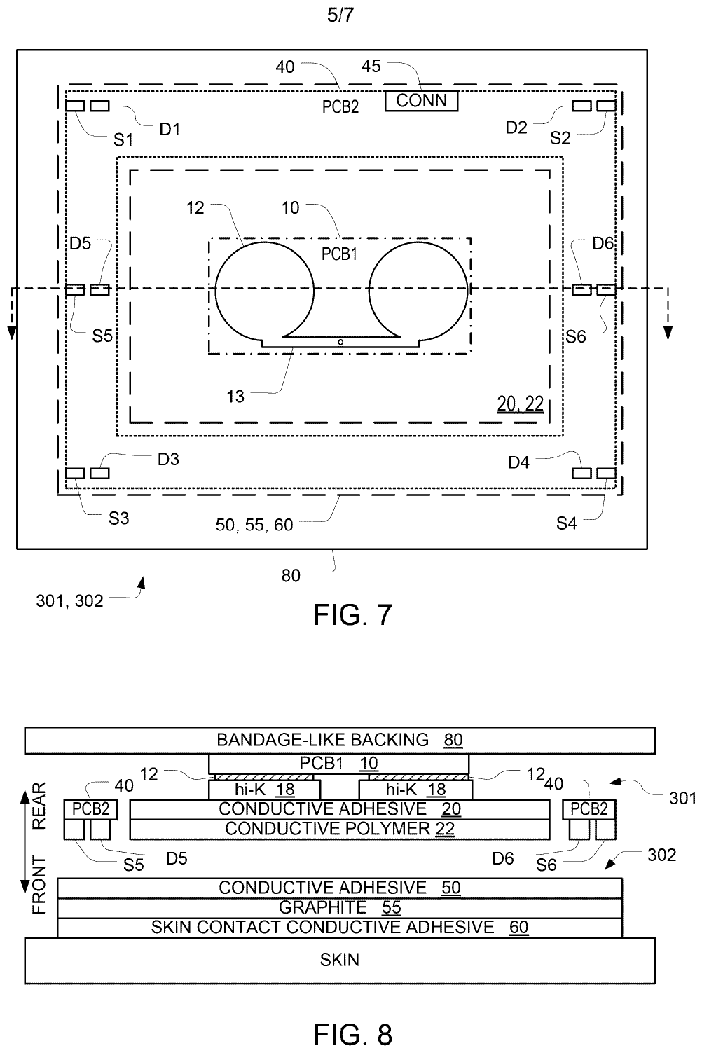

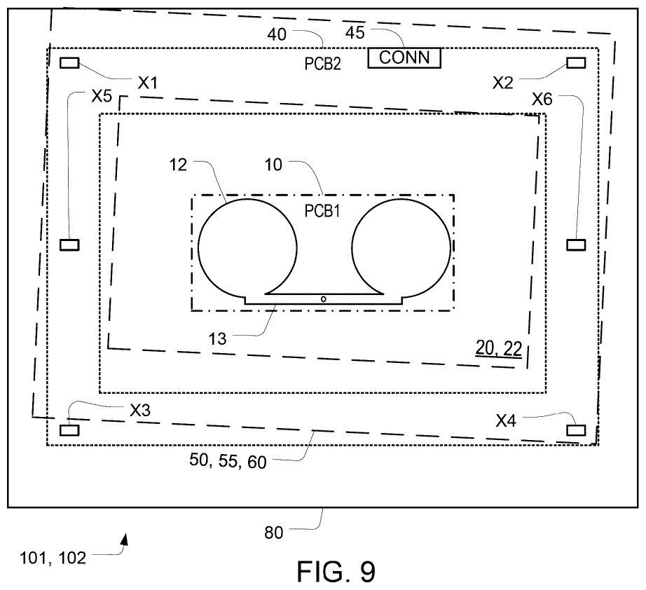

Finally, the resistance-based approach for determining whether the first and second subassemblies have separated from each other described above is not the only approach for determining that the subassemblies have separated. To the contrary, a variety of alternative approaches can be used, including alternative electrical-based approaches (e.g., based on electrical conductance or continuity) as well as optical-based approaches. One example of an optical-based approach for determining whether the first and second subassemblies have separated from each other is described below in connection with FIGS. 7-8.

FIG. 7 is a plan view of a different electrode assembly (e.g., a transducer array) for applying alternating electric fields (e.g., TTFields) to a subject's body that is divided into a first (i.e., rear) subassembly 301 (electrode subassembly) and a second (i.e., front) subassembly 302 (skin-contact subassembly), and FIG. 8 is a section view of the same electrode assembly. During use, the first and second subassemblies 301, 302 will be pressed against each other and in intimate contact. But those two subassemblies 301, 302 are shown spaced apart in FIG. 8 so that the boundary between the two subassemblies will be clear.

In referring to FIGS. 7-8, the figure labelling is similar to that for FIGS. 1-2, except as discussed below. The FIGS. 7-8 embodiment is similar to the FIGS. 1-2 embodiment described above, except that instead of using a set of conductive terminals X1-X6 positioned on the front face of the first subassembly to make resistance measurements, and thereby ascertain whether the first subassembly is in intimate contact with the second subassembly (as described above in connection with FIGS. 1-4), the FIGS. 7-8 embodiment uses an optical approach to ascertain whether the first subassembly 301 is in intimate contact with the second subassembly 302.

A plurality of light sources S1-S6 are positioned (e.g., on the second PCB 40) in the peripheral region of the first subassembly 301, and each of these light sources is aimed in a forward direction (i.e., towards the bottom of the page in FIG. 8). A plurality of light detectors D1-D6 are also positioned (e.g., on the second PCB 40) in the peripheral region of the first subassembly 301, and each of these light detectors is positioned to detect whether light emanating from a respective one of the plurality of light sources S1-S6 is being reflected back by a respective portion of a second subassembly 302. Conductive traces or wires (not shown) are provided between each of the light detectors D1-D6 and each of the light sources S1-S6 and respective pins of the connector 45 to provide the controller 130 (FIG. 3) with access to the light sources and light detectors. Note that while the light sources S1-S6 and the light detectors D1-D6 are located on a different PCBs (PCB 2/PCB 40) than the electrode elements 12 in the embodiment depicted in FIGS. 7-8 (PCB 1/PCB 10), in alternative embodiments (not shown) the light sources S1-S6, the light detectors D1-D6, and the electrode elements 12 could all be located on a single PCB.

The controller ascertains whether the level of light that is detected by each of the light detectors D1-D6 is indicative of the fact that the first subassembly 301 and the second subassembly 302 are in intimate contact at each of the locations of a source/detector pair. This may be accomplished, for example, by positioning each of the light sources S1-S6 so that when it is in intimate contact with the second subassembly 302, all of the light emanating from the light source will be blocked. But as soon as any of the light sources S1-S6 begins to separate from the second subassembly 302, some of the light emanating from those light sources will leak out. And the presence of this leaked-out light (as detected by the light detectors DI-D6) will indicate that the first subassembly 301 and the second subassembly 302 are no longer in intimate contact.

Similar to the situation described above in connection with FIG. 4, if it is determined (based on the levels of detected light) that each of these locations is in intimate contact, the controller will allow the application of alternating electric fields to continue. If, on the other hand, it is determined that at least one of these locations is not in intimate contact, the controller will stop the application of alternating electric fields.

### Section 2—Proper Alignment

In Section 1 above, the hardware depicted in FIGS. 1-2 and in FIGS. 5-6 is described in the context of determining whether the first and second subassemblies 101/102 or 201/202 are either (a) adhered to each other and making good contact with each other or (b) have become separated to the point where one or more of the conductive terminals X1-X6 on the first subassembly is no longer in intimate contact with the second subassembly. The latter condition can be detected, e.g., by determining that the resistance of the path between some (or all) of those terminals has risen above the value that would be expected when the terminals are all making good contact with the second subassembly.

Notably, the exact same hardware can also be used to determine whether the first and second subassemblies 101/102 or 201/202 are properly aligned with each other.

FIG. 9 (with similar figure labelling as that shown in FIG. 1) depicts one example of the spatial relationship between the first subassembly 101 and the second subassembly 102 when those two subassemblies are not aligned with each other. Unlike the situation depicted in FIG. 1 (in which the first and second subassemblies 101, 102 are aligned), those two subassemblies 101, 102 are not aligned in the FIG. 9 example. In this non-aligned example, the conductive terminals X1, X2, X4, X5, and X6 on the first subassembly 101 are all still making good contact with the second layer of conductive adhesive 50 positioned at the rear of the second subassembly 102. But due to the non-alignment, the conductive terminal X3 on the first subassembly 101 does *not* contact the second layer of conductive adhesive 50 positioned at the rear of the second subassembly 102.

Because the conductive terminal X3 on the first subassembly 101 does not contact the second layer of conductive adhesive 50 on the second subassembly 102, the resistance of the path between X3 and all the other terminals X1, X2, X4, X5, and X6 will be above the value that would be expected in the case when all of the terminals X1-X6 are making good contact with the second layer of conductive adhesive 50. The controller can therefore conclude that the second subassembly 102 has either separated from or is not aligned with the first subassembly 101.

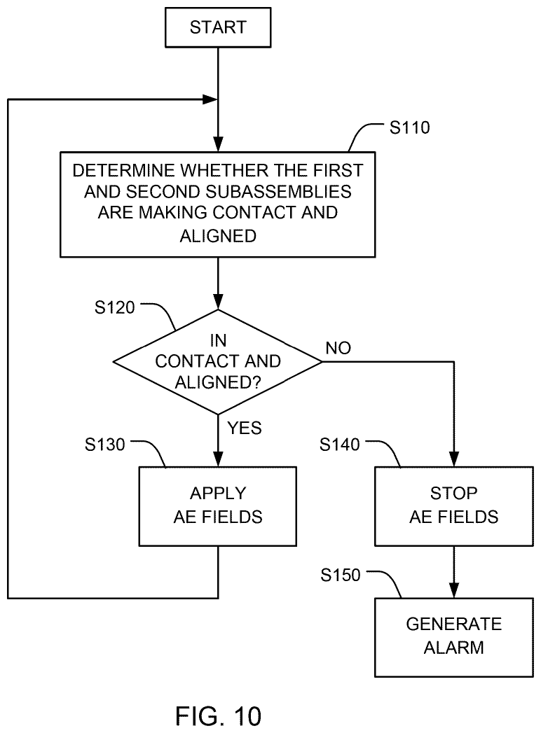

FIG. 10 depicts an example of how the controller 130 (depicted in FIG. 3) can accomplish this. More specifically, in step S110, the controller 130 determines whether the first and second subassemblies 101, 102 are making good contact with each other and aligned (e.g., as described above). Step S120 is a branching step. When a determination has been made in S110 that the first and second subassemblies 101, 102 are making good contact with each other and aligned, the processing flow proceeds to S130, where alternating electric fields (“AE Fields” in FIG. 10) are applied to the subject's body (via the first and second subassemblies 101, 102). On the other hand, when a determination has been made in S110 that the first and second subassemblies 101, 102 are either not making good contact with each other or are not aligned, the processing flow proceeds to S140, where the application of the alternating electric fields is stopped. Optionally, after stopping the alternating electric fields, an alarm or alert (e.g., a visual signal, an auditory signal, or a haptic signal) can be generated in S150 to notify the user that the first and second subassemblies 101, 102 are no longer making good contact with each other or are not aligned. The user can then take appropriate corrective action (e.g., pressing the first and second subassemblies 101, 102 together, re-aligning the first and second subassemblies, or replacing one or both of those subassemblies).

Advantageously, stopping the AC signal that is applied to the electrode assemblies 101/102 when the first and second subassemblies 101, 102 included therein are either no longer making good contact with each other or are not aligned can prevent safety problems from arising.

In some contexts, the controller 130 may be able to determine whether a given high-resistance path arose from a separation of the first and second subassemblies 101, 102 as opposed to a mis-alignment. For example, if the system is operating in a steady-state condition in which low resistance paths exist between all of the terminals X1-X6, and the resistances of all of those paths subsequently increase, then separation of the subassemblies 101, 102 would be the most likely culprit. If the system is operating in a steady-state condition in which low resistance paths exist between all of the terminals X1-X6, and the resistance of one of those paths subsequently increases, then either separation of the subassemblies 101, 102 at one corner or a misalignment of the two subassemblies could be responsible. In other contexts, it may not be possible to distinguish a separation from a mis-alignment (e.g., if the system detects a high resistance path immediately upon power up after the first and second subassemblies 101, 102 have been pressed against each other for the first time). But in this context, distinguishing between the two cases is not necessary because the remedy is the same (e.g., peeling the two subassemblies 101, 102 apart and subsequently pressing them back together again with more care).

The same approach described above in this section for the first and second subassemblies 101/102 can be applied to the first and second subassemblies 201/202 in the FIGS. 5-6 embodiment. Furthermore, the resistance-based approach for determining whether the first and second subassemblies have either (a) separated from each other or (b) are mis-aligned described in this section is not the only approach that can be used. To the contrary, a variety of alternative approaches can be used, including alternative electrical-based approaches (e.g., based on electrical conductance or continuity) as well as optical-based approaches can be used. For example, the same hardware depicted in FIGS. 7-8 can also be used to determine whether the first and second subassemblies 301/302 are either (a) making good contact with each other and aligned or (b) have either become separated or mis-aligned to a point that causes the light levels measured by the light detectors DI-D6 on the first subassembly to change. This can be accomplished by relying on optical signals instead of electrical signals to detect separation or mis-alignment.

While methods of the inventive concept(s) have been described in terms of particular embodiments, variations may be applied to the methods and in the steps or in the sequence of steps of the methods described herein without departing from the scope of the inventive concept(s). In particular, where a method claim does not specifically state in the claims or description that the steps are to be limited to a specific order, it is no way intended that an order be inferred, in any respect.

Headings are provided for convenience only and are not to be construed to limit the invention in any manner. Embodiments illustrated under any heading or in any portion of the disclosure may be combined with embodiments illustrated under the same or any other heading or other portion of the disclosure. Any combination of the elements described herein in all possible variations thereof is encompassed by the invention unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for applying an alternating electric field to a subject's body, the apparatus comprising:
a first subassembly having a front and a rear, wherein the first subassembly includes
a first layer of conductive material positioned at the front of the first subassembly in a central region of the first subassembly, so that a front surface of the first layer of conductive material serves as a front surface of the first subassembly in the central region of the first subassembly,
at least one electrode element positioned between the first layer of conductive material and the rear of the first subassembly, wherein each of the electrode elements has a front surface,
at least one intermediate layer of material positioned between the front surface of each of the electrode elements and a rear surface of the first layer of conductive material, wherein the at least one intermediate layer of material is configured to either (a) capacitively couple each of the electrode elements to the first layer of conductive material or (b) conductively couple each of the electrode elements to the first layer of conductive material, and
a plurality of conductive terminals positioned at the front of the first subassembly in a peripheral region of the first subassembly,
wherein the central region of the first subassembly has a perimeter, and the peripheral region of the first subassembly lies outside the perimeter, and
wherein the first layer of conductive material has an area of at least 10 cm².

2. The apparatus of claim 1, further comprising a second subassembly having a front and a rear,
wherein the second subassembly includes
a second layer of conductive material positioned at the rear of the second subassembly, so that a rear surface of the second layer of conductive material serves as a rear surface of the second subassembly, wherein the second layer of conductive material is shaped and dimensioned to overlap both the central region of the first subassembly and the peripheral region of the first subassembly, and
a third layer of conductive material positioned at the front of the second subassembly in electrical contact with the second layer of conductive material, wherein the third layer of conductive material is configured to adhere to skin,
wherein the front surface of the first layer of conductive material is positioned against the rear surface of the second layer of conductive material when the first subassembly is in contact with the second subassembly, and
wherein the plurality of conductive terminals are positioned against a peripheral region of the second layer of conductive material when the first subassembly is in contact with and aligned with the second subassembly.

3. The apparatus of claim 2, wherein at least one of the second layer of conductive material and the third layer of conductive material is a layer of conductive adhesive or conductive gel.

4. The apparatus of claim 1, wherein the at least one intermediate layer of material comprises (a) a layer of insulating material with a dielectric constant of at least 10 disposed on the front surface of each of the electrode elements, and (b) a first layer of conductive adhesive or conductive gel disposed on, and positioned in front of, the layer of insulating material,
wherein the first layer of conductive material is a layer of conductive polymer,
wherein the first layer of conductive material is disposed on, and positioned in front of, the first layer of conductive adhesive or conductive gel.

5. The apparatus of claim 4, further comprising a second subassembly having a front and a rear,
wherein the second subassembly includes
a second layer of conductive adhesive or conductive gel positioned at the rear of the second subassembly, so that a rear surface of the second layer of conductive adhesive or conductive gel serves as a rear surface of the second subassembly, wherein the second layer of conductive adhesive or conductive gel is shaped and dimensioned to overlap both the central region of the first subassembly and the peripheral region of the first subassembly,
a layer of graphite disposed on, and positioned in front of, the second layer of conductive adhesive or conductive gel, and
a third layer of conductive material disposed on, and positioned in front of, the layer of graphite, and wherein the third layer of conductive material is configured to adhere to skin,
wherein the front surface of the first layer of conductive material is positioned against the rear surface of the second layer of conductive adhesive or conductive gel when the first subassembly is in contact with the second subassembly, and
wherein the plurality of conductive terminals are positioned against a peripheral region of the second layer of conductive adhesive or conductive gel when the first subassembly is in contact with and aligned with the second subassembly.

6. The apparatus of claim 5, wherein the third layer of conductive material comprises a conductive adhesive or a conductive gel.

7. The apparatus of claim 1, wherein the at least one intermediate layer of material comprises a first layer of conductive adhesive or conductive gel disposed on, and positioned in front of, the front surface of each of the electrode elements,
wherein the first layer of conductive material is disposed on, and positioned in front of, the first layer of conductive adhesive or conductive gel, and
wherein the first layer of conductive material is a layer of conductive silicone rubber.

8. The apparatus of claim 7, further comprising a second subassembly having a front and a rear,
wherein the second subassembly includes
a second layer of conductive adhesive or conductive gel positioned at the rear of the second subassembly, so that a rear surface of the second layer of conductive adhesive or conductive gel serves as a rear surface of the second subassembly, wherein the second layer of conductive adhesive or conductive gel is shaped and dimensioned to overlap both the central region of the first subassembly and the peripheral region of the first subassembly,
a layer of graphite disposed on, and positioned in front of, the second layer of conductive adhesive or conductive gel, and
a third layer of conductive material disposed on, and positioned in front of, the layer of graphite, and wherein the third layer of conductive material is configured to adhere to skin,
wherein the front surface of the first layer of conductive material is positioned against the rear surface of the second layer of conductive adhesive or conductive gel when the first subassembly is in contact with the second subassembly, and
wherein the plurality of conductive terminals are positioned against a peripheral region of the second layer of conductive adhesive or conductive gel when the first subassembly is in contact with and aligned with the second subassembly.

9. The apparatus of claim 1, wherein the at least one intermediate layer of material comprises: (a) a layer of insulating material with a dielectric constant of at least 10 disposed on the front surface of each of the electrode elements, (b) a first layer of conductive adhesive or conductive gel disposed on, and positioned in front of, the layer of insulating material, and (c) a layer of graphite disposed on, and positioned in front of, the first layer of conductive adhesive or conductive gel,
wherein the first layer of conductive material comprises a conductive adhesive or conductive gel, and
wherein the first layer of conductive material is disposed on, and positioned in front of, the layer of graphite.

10. The apparatus of claim 9, further comprising a second subassembly having a front and a rear,
wherein the second subassembly includes
a layer of conductive polymer positioned at the rear of the second subassembly, so that a rear surface of the layer of conductive polymer serves as a rear surface of the second subassembly, wherein the layer of conductive polymer is shaped and dimensioned to overlap both the central region of the first subassembly and the peripheral region of the first subassembly, and
a third layer of conductive material disposed on, and positioned in front of, the layer of conductive polymer, and wherein the third layer of conductive material is configured to adhere to skin,
wherein the front surface of the first layer of conductive material is positioned against the rear surface of the layer of conductive polymer when the first subassembly is in contact with the second subassembly, and
wherein the plurality of conductive terminals are positioned against a peripheral region of the layer of conductive polymer when the first subassembly is in contact with and aligned with the second subassembly.

11. The apparatus of claim 1, wherein each of the conductive terminals comprises a conductive pad of a printed circuit.

12. The apparatus of claim 1, wherein the plurality of conductive terminals comprises at least four conductive terminals, and
wherein the apparatus further comprises a circuit configured to measure resistances or electrical continuities between a plurality of pairs of the at least four conductive terminals.

13. The apparatus of claim 12, wherein the circuit is further configured to generate an output when the measured resistances or electrical continuities deviate from values that would be expected when all of the conductive terminals are making good contact with a sheet of conductive material.

14. The apparatus of claim 13, wherein the circuit is further configured to disable operation of the first subassembly in response to the generation of the output.

15. The apparatus of claim 14, wherein the at least four conductive terminals comprises a first conductive terminal positioned near a first corner of the first subassembly, a second conductive terminal positioned near a second corner of the first subassembly, a third conductive terminal positioned near a third corner of the first subassembly, and a fourth conductive terminal positioned near a fourth corner of the first subassembly.

16. The apparatus of claim 1, wherein the plurality of conductive terminals comprises at least three conductive terminals, and
wherein the apparatus further comprises a circuit configured to measure resistances or electrical continuities between a plurality of pairs of the at least three conductive terminals.

17. A method of inhibiting or preventing the operation of a system for applying alternating electric fields to a subject's body, the system including a second subassembly that is configured for positioning on the subject's body and a first subassembly that is removably affixed to the second subassembly, the method comprising:
applying alternating electric fields to the subject's body via the first subassembly and the second subassembly when the first subassembly is affixed to the second subassembly;
determining whether the first subassembly and the second subassembly are making good contact with each other; and
stopping the applying of the alternating electric fields to the subject's body when a determination has been made that the first subassembly and the second subassembly are not making good contact with each other.

18. The method of claim 17, further comprising providing at least one of a visual signal, an auditory signal, and a haptic signal to indicate that the first subassembly and the second subassembly are not making good contact with each other.

19. The method of claim 17, wherein the determining comprises measuring at least one resistance or electrical continuity between a plurality of conductive terminals positioned on the first subassembly, and wherein each of the resistance or electrical continuity measurements travels through a component in the second subassembly.

20. A method of inhibiting or preventing the operation of a system for applying alternating electric fields to a subject's body, the system including a second subassembly that is configured for positioning on the subject's body and a first subassembly that is removably affixed to the second subassembly, the method comprising:
applying alternating electric fields to the subject's body via the first subassembly and the second subassembly when the first subassembly is affixed to the second subassembly;
determining whether the first subassembly and the second subassembly are making good contact with each other and aligned with each other; and
stopping the applying of the alternating electric fields to the subject's body when a determination has been made that the first subassembly and the second subassembly are either not making good contact with each other or not aligned with each other.

21. The method of claim 20, further comprising providing at least one of a visual signal, an auditory signal, and a haptic signal to indicate that the first subassembly and the second subassembly are either not making good contact with each other or not aligned with each other.

22. The method of claim 20, wherein the determining comprises measuring at least one resistance or electrical continuity between a plurality of conductive terminals positioned on the first subassembly, wherein each of the resistance or electrical continuity measurements travels through a component in the second subassembly, and
wherein the plurality of conductive terminals are positioned on the first subassembly so that electrical continuity is interrupted when the first subassembly and the second subassembly are not aligned with each other.

23. The method of claim 20, wherein the determining comprises:
illuminating a plurality of light sources positioned on the first subassembly, wherein each of the plurality of light sources is aimed towards the second subassembly; and
detecting, using a plurality of light detectors positioned on the first subassembly, how much light from the plurality of light sources has arrived at the plurality of light detectors.
